(19) United States
(12) Patent Application Publication
(10) Pub. No.: US 2008/0119832 A1
(43) Pub. Date: May 22, 2008

(54) Multi-Modal Scanning Confocal Adaptive-Optic Macroscope System and Associated Methods

(75) Inventor: Paul J. Cronin, Allen, TX (US)

Correspondence Address: HAYNES AND BOONE, LLP, 901 Main Street, Suite 3100, Dallas, TX, 75202, US

(73) Assignee: MOLTEN LABS, INC., Allen, TX (US)

(21) Appl. No.: 11/941,822

(22) Filed: Nov. 16, 2007

Related U.S. Application Data
(60) Provisional application No. 60/866,200, filed on Nov. 16, 2006.

(51) Int. Cl. A61B 18/18

(52) U.S. Cl. 606/15

(57) ABSTRACT

In some embodiments, the present disclosure is directed to a multi-modal, scanning, confocal, adaptive-optic macroscope system and related methods of use. Embodiments of the present disclosure improve upon existing photodynamic therapy methods and compete with more invasive oncology procedures (such as surgery, radiation, and/or chemotherapy) by providing deeper light penetration within the human body, giving medical personnel the ability to treat forms of cancer previously untreatable with existing photodynamic therapies, and with less damage to healthy tissue than current procedures. Embodiments of the present disclosure may be used for other medical purposes including internal artery and vein cauterizing (photo-coagulation), laser pulse induced sonication for dissolving kidney stones, removal of birth marks or tattoos, and/or for photo-biostimulation.

### FIELD/BACKGROUND

This utility application claims priority to U.S. provisional application Ser. No. 60/866,200, filed on Nov. 16, 2006.

The present disclosure is directed to improved instrumentation and methods for performing various types of photodynamic therapy. More particularly, one aspect of the present disclosure is directed to a scanning, confocal, adaptive-optic macroscope system for use in the imaging and/or treatment of deep tissue oncology, dermatology, and other medical conditions. In some instances the scanning, confocal, adaptive-optic macroscope system is configured for use with known photodynamic therapy vectors (e.g, drugs) as well as nanophosphor markers.

Photodynamic therapy involves the use of certain wavelengths of light to activate photosensitive drugs or heat nanoparticles, either of which can be bound to cancer cells. Once activated, these materials kill the cancerous cells to which they are attached. While the known instrumentation and methods have been adequate for their intended purposes, they have not been satisfactory in all respects.

Therefore, there remains a need for improved instrumentation and methods for performing photodynamic therapies.

### SUMMARY

The present disclosure relates to photodynamic therapy methods and devices.

In one aspect, the present disclosure provides a macroscope system for use in deep tissue photodynamic therapy. The system includes an energy source for providing a point source of energy. The energy source is configured for providing a point source of energy having a first characteristic for imaging the deep tissue and a second characteristic for treating the deep tissue. The system also includes an adaptive optic device in optical communication with the point source of energy and an optical head portion in optical communication with the point source of energy. The optical head portion comprising a focal lens moveable along an optical axis for adjusting a depth of a focal point of the energy within the patient's tissue. The adaptive optic device of the system is configured to at least partially correct for aberrations caused by patient tissue positioned between the focal lens and the deep tissue.

In another aspect, the present disclosure provides a method of performing deep tissue photodynamic therapy. The method includes introducing a photodynamic therapy vector into a region of interest within a deep tissue of a patient and imaging the region of interest with a scanning, confocal macroscope to identify one or more cancerous portions of the region of interest. The method also includes activating the photodynamic therapy vector within the one or more cancerous portions of the region of interest with the scanning, confocal macroscope. In some embodiments, the imaging comprises forming a 3-D image of the region of interest. In some embodiments, the scanning, confocal macroscope utilizes adaptive optics to correct aberrations caused by the patient's tissue during imaging. In some embodiments, the imaging comprises emitting a laser from the macroscope at a first level of intensity and the activating comprises emitting a laser from the macroscope at a second level of intensity, where the second level of intensity is greater than the first level of intensity. In some embodiments, the macroscope is switched from the imaging mode and the first level of intensity to the activating mode and the second level of intensity immediately upon identifying a portion within the region of interest.

In another aspect, the present disclosure provides a macroscope system for use in deep tissue photodynamic therapy. The system includes a single-mode fiber optic cable for providing a point source of light and a plurality of lasers optically connected to the fiber optic cable. Each of the plurality of lasers is configured for emitting a light of a different wavelength into the fiber optic cable. An adaptive optic device is in optical communication with the point source of light and a tip mirror is in optical communication with the adaptive optic device. A tilt mirror is in optical communication with the tip mirror and an optical head portion in optical communication with the tilt mirror. The optical head portion is configured for emitting a beam of light having a focal point within the deep tissue of a patient. The optical head portion includes a focal lens moveable along an optical axis for adjusting the depth of the focal point of the energy within the deep tissue. The adaptive optic device is configured to at least partially correct for aberrations caused by patient tissue between focal lens and the deep tissue. The macroscope system is configured to image the deep tissue of the patient in a first mode and to excite a photodynamic therapy vector within the deep tissue of the patient in a second mode.

In another aspect, the present disclosure provides a patient treatment table. Table is sized to allow a patient to sit or lay down on the table and includes a window. In use, the treatment area or area of interest on the patient is positioned adjacent to the window. In some embodiments, the window comprises a glass panel providing visual access to a lower portion of the table housing an imaging and treatment device. In some embodiments, the imaging and treatment device is a scanning, confocal, adaptive-optic macroscope system as described with respect to other embodiments of the present disclosure. In some instances, the output or optical head of the macroscope is positioned adjacent to the window of the table. In some instances the table is particularly suited for treating breast cancer. For example, in some embodiments the treatment table is configured for a patient to lay face-down such that the patient's breasts are positioned adjacent to the window may include a device to limit movement of the patient's breasts relative to the table during imaging and treatment.

In some embodiments, the present disclosure is directed to a multi-modal, scanning, confocal, adaptive-optic macroscope system and related methods of use. Embodiments of the present disclosure improve upon existing photodynamic therapy methods and compete with more invasive oncology procedures (such as surgery, radiation, and/or chemotherapy) by providing deeper light penetration within the human body, giving medical personnel the ability to treat forms of cancer previously untreatable with existing photodynamic therapies, and with less damage to healthy tissue than current procedures. Embodiments of the present disclosure may be used for other medical purposes including internal artery and vein cauterizing (photo-coagulation), laser pulse induced sonication for dissolving kidney stones, removal of birth marks or tattoos, and/or for photo-biostimulation.

Further aspects, forms, embodiments, objects, features, benefits, and advantages of the present disclosure shall become apparent from the detailed drawings and descriptions provided herein.

### BRIEF DESCRIPTION OF THE DRAWINGS

FIG. 1 is a diagrammatic schematic illustration of a macroscope system according to one embodiment of the present disclosure.

FIG. 2 is a diagrammatic schematic illustration of a macroscope system according to another embodiment of the present disclosure.

FIG. 3 is a diagrammatic schematic illustration of the macroscope system of FIG. 2 treating a cancerous region of a patient according to one embodiment of the present disclosure.

FIG. 4 is a flowchart illustrating a method according to one embodiment of the present disclosure.

FIG. 5 is a flowchart illustrating a method according to one embodiment of the present disclosure.

FIG. 6 is a diagrammatic schematic illustration of a patient treatment table incorporating a macroscope system according to one embodiment of the present disclosure.

### DESCRIPTION OF THE PREFERRED EMBODIMENTS

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications in the described devices, instruments, methods, and any further application of the principles of the disclosure as described herein are contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure.

Currently, there are two main methods of photodynamic therapy—(1) photothermal ablation and (2) photosensitizing. The first method—photothermal tumor ablation—kills cancer cells by raising their temperature above a survivable limit. This is achieved by the incident or activating light being absorbed by inorganic nanoparticles conjugating or bound to the cancerous cells. The cancer cells bound to the nanoparticles absorb more of the laser light than the surrounding healthy cells raising the cancerous cell mortality by a factor of 2-to-1 relative to the healthy cells. In some instances, the binding between the nanoparticles and the cancer cells is achieved utilizing the fact that many cancer cells have a protein known as Epidermal Growth Factor Receptor (EGFR) covering their surface. Healthy cells typically express far less of this protein compared to cancer cells. By conjugating gold nanoparticles to an antibody for EGFR (anti-EGFR) the gold nanoparticles may be attached to the cancer cells. There are detectable differences between the malignant cancer cells and the normal cells when bound to the gold nanoparticles in terms of light scattering and absorption spectra. For example, when bound to the gold nanoparticles, the cancer cells scatter light making it easier to differentiate the non-cancerous cells from the cancer cells. Halas and colleagues have shown that nanoshells are about 10,000 times more effective at Surface-Enhanced Raman Scattering (SERS) than traditional methods.

Further, changing the shape of the particle at the nanoscale allows engineers and scientists to modify the properties of the associated plasmon waves to control the way that the metal nanostructure responds to light. A spherical nanoparticle is not necessarily the best morphology for photothermal ablation. A team of researchers at Rice University have found that elongated nanostructures have a unique set of optical properties that could be useful in various cancer research applications. The researchers have developed gold “nanorice,” a layered structure of gold and iron that looks like a nanoscale piece of rice. The researcher's investigations found that nanorice possesses far greater structural tenability than nanoshells as well as another commonly studied optical nanoparticle, the nanorod. In fact, tests indicate that nanorice is the most sensitive surface plasmon resonance (SPR) nanosensor yet.

The second method—photosensitizing photodynamic therapy—kills the cancer cells by first administering a photosensitizer to the patient and then exposing the cancer cells to a light or wavelength of energy suitable for exciting the photosensitizer. Consider, for example, a photodynamic therapy for the treating basal cell carcinoma (BCC) using the photosensitizing method. BCC is the most common form of skin cancer in humans. Other non-photodynamic treatments for BCC involve surgical excision, cryogenic treatment with liquid nitrogen, or localized chemotherapy. Using a photosensitizing photodynamic treatment, a photosensitizer (such as the vector Photofrin) is injected into the treatment area. The Photofrin is taken up by the cancer cells, which then convert the Photofrin into a photosensitizer, such as protoporphyrin IX. The treatment area is then illuminated with an activating light. In some instances, the exposure lasts less than an hour. The protoporphyrin IX absorbs the light, exciting it to an excited singlet state. Intersystem crossing occurs, resulting in excited triplet protoporphyrin IX. Energy is transferred from triplet protoporphyrin IX to triplet oxygen, resulting in singlet (ground state) protoporphyrin IX and excited singlet oxygen. The resultant singlet oxygen reacts with biomolecules, killing the cells in the treatment area. Within a few days, the exposed skin and carcinoma will scab and flake away. In a few weeks, the treated area will have healed, leaving the healthy skin behind. For extensive malignancies, repeat treatments may be required.

While many achievements have been made in the materials arena of nanomedicine, there has been a lack of development in the associated devices utilized to implement these therapies to support an expanded use of these medicines. For example, photodynamic therapy currently allows for the treatment of certain surface cancers, but at a very limited, if any, penetrative depth. Currently, some of the most common cancers (e.g., breast and thyroid cancers) currently cannot be treated with photodynamic therapy. There are several limitations of present photodynamic therapy methods.

First, current photodynamic therapy methods have severe penetration depth limitations. The present day use of photodynamic therapy for oncology treatment is limited due to the inherent optical problem of light traveling within human tissue. The necessary light required for activating the photodynamic therapy drugs rarely travels strictly to the target cells. Rather, the illuminating light diffuses very quickly as it enters the patient's tissue. This causes the surrounding healthy cells to be negatively affected while the reduced intensity of light at the marked cancerous cells minimizes the efficacy of the photodynamic therapy drugs. Accordingly, the application of photodynamic therapies has been limited to the treatment of tumors on or just under the skin, or on the lining of some internal organs that are accessible through the use of endoscope or by cutting the patient open. For these same reasons, conventional photodynamic therapy methods are less effective or ineffective in treating large tumors and metastasis.

Further, current photothermal ablation techniques are limited by the precision of the treatment. In particular, the metal/metal dielectric nanoparticles are limited in their ability to kill only the cancerous cells. Accordingly, all of the illuminated cells, both cancerous and healthy, bound with gold nanoparticles are likely to be killed during the treatment. While the cancer cells have a mortality rate twice that of the healthy cells using current treatment methods, the treatment methods still result in significant mortality of healthy cells.

Further, current photodynamic therapy methods require a substantial delay between the medicinal treatment and the application of the light or energy source. Cancerous cells have vascular leakage. That is, the contents of the cancer cells literally leak into the surrounding area. This reduces the rate of removal of the existing photodynamic therapy vectors, such as Photofrin, from the cancerous cells relative to the of the body's normal removal rate by healthy cells. Consequently, after a delay of 24 to 72 hours, there is more PhotoFrin surrounding the cancerous cells than the healthy cells. Currently, there is a 24 to 72 hour delay between application of the drug and photodynamic treatment. This to allow for the removal of a substantial amount of the Photofrin from healthy cells by the body's natural processes. However, not all Photofrin will be been removed from the healthy cells. Even after the delay, the remaining Photofrin in the healthy cells can cause significant detriment to the patient during the treatment, causing swelling and/or cell mortality.

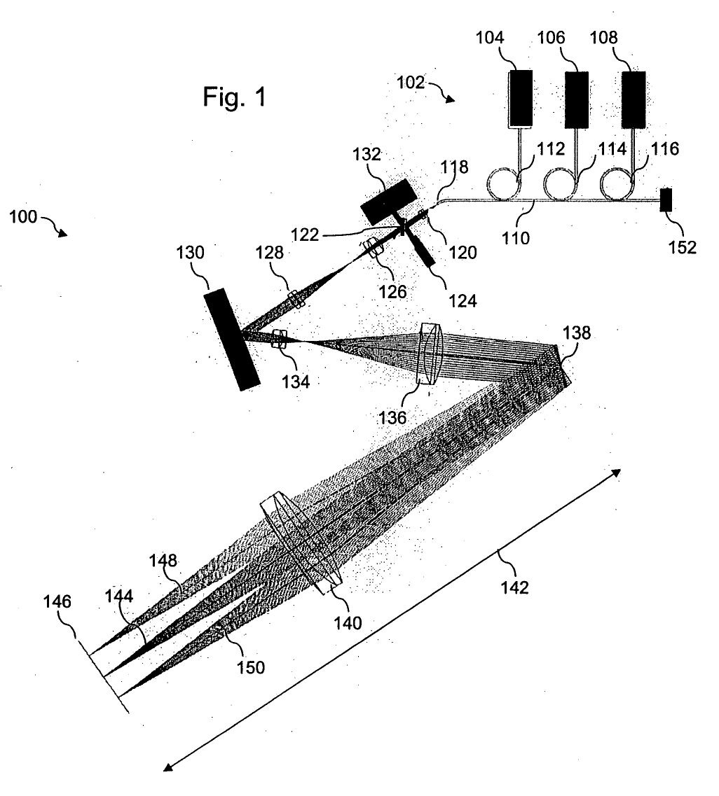

Referring now to FIG. 1, shown therein is a macroscope system 100 according to one aspect of the present disclosure. The difference between the macroscope system 100 and a microscope should be noted. Whereas a microscope is capable of imaging only a few cells of a tumor, the macroscope system 100 in accordance with the present disclosure is capable of imaging and treating whole tumors. In particular, the macroscope system 100 has an image field between 2-20 cm². In one particular embodiment, the macroscope system has an image field of with a height and width of approximately 4.0 cm and a depth of approximately 1.5 cm. Changing the various lenses, mirrors, and/or other aspects of the system results in a bigger or smaller image field or field of view. Further, within a single embodiment the image field may be increased or decreased. However, the resolution of the image may be harmed by increasing the image field in some instances.

The system 100 includes an energy source 102. The energy source 102 is utilized by the system 100 for imaging purposes as well as treatment purposes. In the current embodiment, the energy source 102 includes a plurality of lasers 104, 106, and 108. Each of the lasers 104, 106, 108 comprises a different wavelength of light. In other embodiments, the energy source 102 includes more or less lasers. In some embodiments, the energy source 102 includes a single laser. In lieu of or in addition to the lasers 104, 106, and 108 the energy source 102 comprises one or more LEDs in other embodiments. In some embodiments, lasers with a wavelength between 550 nm and 1550 nm are used. In some embodiments, at least one of the lasers includes a wavelength of approximately 850 nm. In some embodiments, at least one of the lasers includes a wavelength of approximately 632 nm. In one particular embodiment, at least one of the lasers comprises a HeNe laser having a wavelength of approximately 632 nm. The specific wavelengths chosen and the number of different wavelengths chosen for the energy source 102 are selected based on the photodynamic therapy vectors in some instances.

It is contemplated that any number and combination of energy sources may be utilized based on the photodynamic therapy vectors utilized. In that regard, the variability of the available energy sources allows for combinations of energy sources to be used to better image and/or treat the cancerous region. For example, in some imaging modes more than one specific wavelength of light is needed to differentiate the marker from the surrounding cells. In some instances, other wavelengths are needed for their penetrative ability. In some embodiments, still other wavelengths are needed to separate out multiple markers in the field—each marker being wavelength specific. To utilize these multi-spectral approaches the optical system must support these myriad of needs as permitted by the energy source 102.

Embodiments of the present disclosure address these needs by using the end of an optical fiber (e.g., a single mode fiber or a multi-mode fiber) as a pinpoint source of light (necessary for confocal imaging). However, much technology already exists for introducing multiple sources of light (lasers, LEDs, incandescent lamps, flash lamps, etc.) into the other end of the fiber. This gives the flexibility to work with not just one photodynamic therapy technique, but to have the ability to choose a suitable system for different applications from multiple current and future techniques.

Each of the lasers 104, 106, 108 are optically connected to a fiber optic cable 110 via connectors 112, 114, 116, respectively. In the current embodiment, the fiber optic cable 110 is a single-mode fiber. In some embodiments, the single-mode fiber has a core diameter between 1 μm and 10 μm. In other embodiments, a multi-mode fiber is used. Connectors 112, 114, 116 are optical connectors utilized to introduce the laser light emitted from the lasers 104, 106, 108 into the fiber optic cable 110. In the present embodiment, the connectors 112, 114, 116 are optical circulators. In other embodiments, the connectors 112, 114, 116 include other types of optical connectors or switches. Generally, any connector cable of introducing the energy from the energy source 102 into the cable 110 may be used in some embodiments. The lasers 104, 106, 108 are connected the fiber optic cable 110 such that one or more of the lasers may introduce light in to the cable. That is, a single laser may be activated or multiple lasers may be activated simultaneously. The determination of which lasers are to be activated and when is driven by such factors as the photodynamic therapy vectors being utilized, whether imaging or treatment is being performed, physician preference, and/or other factors.

After the light has been introduced into the cable 110 from the energy source 102, the light is emitted from the end 118 of the fiber optic cable 110. The light exiting from the fiber optic cable 110 is the point source of light for the system 100. In some embodiments, the light is emitted from the fiber optic cable 110 with a half angle between approximately 20-30 degrees. The light emitted from the fiber optic cable 110 then passes through a lens 120 before reaching a splitter 122. The lens 120 substantially collimates the light in some instances. In some embodiments, the splitter 122 comprises a beam splitter. Further, in some instances the beam splitter comprises a polarizing beam splitter. In other embodiments, the splitter 122 comprises a pickoff mirror. Further, in some instances the pickoff mirror picks off between 1-10% of the light passing therethrough. As the light passes through the splitter 122, some of the light is directed to a beam dump 124. The beam dump 124 absorbs the light that is directed its way. In that regard, the beam dump 124 may be utilized to reduce or eliminate any unwanted reflection from the energy source 102.

The remainder of the light passing through the splitter 122 continues on through a lens 126 and a lens 128 before reaching an adaptive optic device 130. In some embodiments, the adaptive optic device 130 comprises a deformable mirror. In other embodiments, the adaptive optic device 130 comprises a MEMS device. Generally, the adaptive optic device 130 may be any suitable adaptive optics module including bimorph, piezo, LCD, MEM, or otherwise. The adaptive optic device 130 is utilized to reduce the effects of optical distortion caused by human tissue, defects in the lenses/mirrors of the system 100, and/or other factors by measuring or monitoring the distortion caused by the tissue and rapidly compensating for it. The aberrations caused by human tissue are detrimental to normal imaging procedures, limiting imaging to depths within the tissue to a few microns. Whereas, embodiments of the present disclosure such as the system 100 allow imaging and treatment cancerous cells at depths up to 1 cm in depth. In other embodiments of the present disclosure, imaging and treatment may be performed at depths greater than 1 cm. In some instances, embodiments of the present disclosure are utilized for surface treatments. In this manner, the adaptive optic device 130 along with the other components of the system 100 are utilized to minimize the scattering of light within human tissue, increase the penetration depth of the light, and increase drug efficacy while simultaneously reducing the required illumination intensity to activate the drugs.

The adaptive optic device 130 is controlled at least in part by a wavefront detector 132. The wavefront detector 132 detects the distortion by monitoring the light reflected back through the system. In some embodiments, the wavefront detector 132 is a Shack-Hartmann sensor. Based on the detected distortion of the wavefront detector 132, an appropriate correction to the adaptive optic device (e.g., adjustment of the deformable mirror) can be determined. In some embodiments, a computer (not shown) connected to the waverfront detector 132 is utilized to calculate an optimum mirror shape for the adaptive optic device 130 based on the detected distortion. The computer then directs the deformable mirror of the adaptive optic device 130 to reshape itself accordingly. In this manner the adaptive optic device 130 may be continuously updated to accommodate for the detected distortion. In some embodiments, the adaptive optic device 130 is updated in an interval between 1-10 milliseconds. In some embodiments, the adaptive optic device 130 and the system 100 in general are configured to operate utilizing a pulse-echo mode. In other embodiments, the system is configured to operate in a continuous wave mode. Accordingly, the energy source 102 and the lasers 104, 106, and 108 are configured for emitting light in a pulse mode and/or a continuous wave mode in some embodiments.

The light is reflected off of the adaptive optic device 130 through a lens 134 and a lens 136 before reaching a tip and tilt mirror 138. The tip and tilt mirror 138 is utilized to control a two-dimension position of the light beam emitted from the system 100. In some embodiments, the tip and tilt mirror 138 deflects the beam of light to sweep the beam of light laterally and vertically through the skin tissue. In some instances, the tip and tilt mirror 138 is controlled by a computer (not shown) in communication with the adaptive optic device 130 and the wavefront detector 132. In some embodiments, the tip and tilt mirror 138 comprises a confocal scanning tip-tilt mirror such as the Ball Aerospace BSM45, which can be found at <http://www.balaerospace.com/fsm_BSM45.html>.

After being reflected off of the tip and tilt mirror 138, the light continues through a lens 140. The lens 140 controls the depth of the focal point 146 of the beam 144 as it exits the system 100. In that regard, the tip and tilt mirror 138 may be understood to control the position of the focal point of the beam in an x-y plane (as indicated by alternate beams 148 and 150), whereas the lens 140 is utilized to control the depth of the focal point 146 along a z-axis perpendicular to the x-y plane. In that regard, the lens 140 may be moved or translated along an axis 142 parallel to the z-axis to control the depth of the focal point 146. In some embodiments, the lens 140 is translated by an actuator controlled by the computer that is also in communication with the adaptive optic device 130, the wavefront detector 132, and the tip and tilt mirror 138. The motion of the focusing lens 140 controls the depth of penetration of the focal point 146 formed by the adaptive optics and confocal elements. The translating optical head module or lens 140 allows for the light to be focused at different depths within the tissue, including depths deep enough to treat at least some forms of breast cancer and other deep tissue cancers. In this regard, deep tissue in the context of the present disclosure is understood to be any subsurface tissue of the patient. Accordingly, deep tissue includes tissue ranging from a depth of 50 microns to 20 mm from an accessible surface. The finely tuned focal point 146 of the beam 144 is swept laterally and vertically through the tissue, allowing for a point-by-point formation of an image of the patient's tissue.

In some embodiments, the focusing lens 140 is part of an optical head component of the system 100. In that regard, the optical head component is considered to be the set of optics between the patient's tissue and the tip and tilt mirror 138. In some embodiments, the optical head component includes a static negative optical element (divergent), a static positive optical element (convergent), a static or dynamic aperture, and a dynamic positive optical element. Note that each of these elements may in fact be a set of optical elements such that certain aberrations (e.g., chromatic) are corrected. The combination divergent element and the convergent element acts as an angle multiplier for the tip and tilt mirror. This allows for very small angular movements of the tip and tilt mirror to be magnified. The particular amount of angular magnification is determined by the magnification of the positive and negative optical elements acting in unison. The aperture—static or dynamic—allows for control of the amount of light impinging the skin and control over the numerical aperture of the final light entering the tissue. The dynamic positive optical element converges the collimated beam of light passing through the aperture down to the focus point or active volume within the tissue. This finely focused region is at least partially defined controlled by the adaptive optical elements of the system. By moving the positive optical element along the optical axis 142, the penetration depth of the focus spot into the tissue is controlled. In some embodiments, the focal depth is controlled only by the use of the adaptive optic device 130 and the tip and tilt mirror 138 and the positive optical element is substantially static.

The focal point 146 of the system 100 results in a “high NA system.” That is, the light defined by the beam 144 that collapses down to the point has a large internal angle such that there is a very high energy density at the focal point 146, but everywhere else outside of the focal point there is energy density is substantially reduced. In particular, the energy outside of the focal point 146 is insufficient to activate the photodynamic therapy vectors in some instances. Accordingly, only the photodynamic therapy vectors of the targeted or marked cells will be activated in subsequent treatment steps. Thus, healthy cells surrounding the targeted cancerous cells will not be affected. It should be noted that while the focal point 146 is illustrated as a single point, the active volume surrounding the focal point—the volume with sufficient intensity to activate the photodynamic therapy vectors—is not a single point. Rather, in some embodiments the active volume is at least partially elongated along the z-axis. In some instances the active volume has dimensions generally within the following ranges: a length between 100-1000 nm, a width between 20-500 nm, and a thickness between 20-500 nm. In some instances, the active volume is not a single, contiguous region. Rather, in some instances the active volume is defined by a plurality of discrete regions that together define the active volume.

Some of the light reflects back from the focal point 146 through the system 100. In particular, the reflected light passes through the lens 140, reflects off of the tip and tilt mirror 138, passes through lenses 136 and 134, reflects off of the adaptive optic device 130 and through lenses 128 and 126 before reaching the splitter 122. More specifically, as the reflected light passes over the adaptive optic device 130, the adaptive optic device “undoes” the aberrations of the reflected light coming up through the tissue and system 100. These aberrations are equal to the aberrations faced by the light on the way down to the focal point 146. At the splitter 122, a portion of the reflected light is directed to the wavefront detector 132. This part of the light returning from the tissue is “picked-off” and sampled by the wavefront detector 132 to constantly monitor the wavefront of the reflected light. The wavefront of the reflected light is utilized to determine the exact equal and opposite aberrations needed to be intentionally introduce by the adaptive optic device 130 (and/or the tip and tilt mirror 138) to cancel out the aberrations. This wavefront information and/or the corresponding changes of required of the adaptive optic device 130 are continually fed to the adaptive optic device.

Due to the adaptive optic device 130 completely reversing the aberrations of the optical system (including aberrations caused by the lenses and mirrors of the system 100 in addition to the tissue), the returning light falls back upon, and is collected by, the optical fiber 110. In some embodiments, a small portion of the reflected light is also directed to the beam dump 124. In other embodiments, none of the reflected light is directed to the beam dump 124. The remainder of the reflected light continues through lens 120 and back towards the optical fiber 110. The fiber 110 only receives light from the focal spot 146 within the tissue. Light from adjacent areas in the tissue misses the optical fiber entrance aperture (which is on the order of 5-10 μm in some embodiments) and, therefore, is not detected. This rejection of out of focus light is at least partially achieved through the use of the confocal system. Embodiments of the present disclosure, including the current system 100, comprise a macroscope having confocal elements. The confocal aspects of the system 100 and other embodiments of the present disclosure provide the ability to control depth of field, eliminate or reduce background information away from the focal plane that typically leads to image degradation, and/or collect serial optical sections from turbid specimens. In some instances, the confocal aspects of the systems use spatial filtering techniques to eliminate out-of-focus light and/or glare in specimens whose thickness exceeds the immediate plane of focus.

The collected light travels back along the same fiber optic cable 110 as the initial light from the energy source 102 enters the system 100. At the other end of the cable 110, the return light is separated from the input light and is received by the optical detector 152. The reflected light sensed by the optical detector is utilized to form the images of the patient's tissue. In some embodiments, the optical detector 152 is in communication with a computer for producing 2-D and 3-D renderings of the detected images. In some embodiments, the optical detector 152 is a high speed optical detector. In some embodiments, the light received by the optical detector 152 is filtered by optical filters into separate wavelength bands where each band falls upon a separate telecom detector. In that regard, the optical detector 152 comprises a plurality of optical detectors in some instances. In some instances, the various wavelengths and/or optical detectors are utilized for monitoring Raman scattering, fluorescence, absorption, and/or upconversion. In that regard, Raman scattering and absorption allow for the detection of gold nanoparticles. By analyzing the light received the optical detector 152, in addition to or in lieu of imaging the patient's tissue it can be determined if there has been upconversion of nanophosphor powders, absorption by gold nanoparticles, and/or fluorescence of PhotoFrin.

In some embodiments the reflected light detected by the optical detector 152 is screened to better clarify the resulting images. For example, in some embodiments the optical detector 152 is configured such that it accepts a majority of the ballistic reflected light and rejects a majority of the multiply scattered reflected light. In that regard, the timing of the optical detector 152 is coordinated with the emission of light from the energy source 102 such that the optical detector accepts reflected light only within a predetermined time period after the emission of the light. The predetermined time period is chosen such that it coincides with the timing of the ballistic reflected light reaching the optical detector 152. In some embodiments, the predetermined time period is determined by a computer in communication with the optical detector 152, adaptive optic device 130, wavefront detector 132, tip and tilt mirror 138, and/or the lens 140. Reflected light received outside of the predetermined time period, which may consist of a substantial portion of multiply-scattered reflected light, is rejected or ignored by the optical detector 152 and/or the computer utilized for producing the images based on the reflected light. In one particular embodiment, the emission of light from the energy source 102 is between approximately 0.5-5.0 picoseconds and the associated predetermined time period is between approximately 1-10 nanoseconds. While the term predetermined has been used to describe the time period it is understood that this is merely exemplary and should not be considered limiting. For example, in some embodiments an appropriate time period is determined in near real time. In other embodiments, the time period may be determined after reception of the reflected light and taken into account when producing the image.

While the term reflected light has been utilized in describing the light that returns through the system for imaging and adaptive optics purposes, it is understood that this is merely exemplary and not limiting. That is, in some instances the light returning through the system 100 is not necessarily reflected light. Rather, in some embodiments the light is scattered. In other embodiments, the light is a fluorescence from an excited element, such as Photofrin. Accordingly, it is understood that in some embodiments the systems described herein utilize light that is not reflected for imaging and adaptive optics purposes.

The system 100 focuses a beam 144 at a focal point 146 within the patient's tissue and then sweeps this focal point over the defined area of interest. The defined area of interest is determined by a treating physician in some instances. In other instances, the defined area of interest is determined by a computer or other device based on prior and/or current imaging results. The system 100 sweeps the beam over the area of interest to both image the area (using a low laser power) and treat the area (using a high laser power) by killing the cancerous cells. In this manner, the system 100 provides a multi-modal, scanning, confocal, adaptive-optic macroscope system. In some embodiments, the lenses utilized in the system 100 are off-the-shelf IT lenses. In one particular embodiment, the lenses include one or more of Edmund Optic lenses, parts 45784, 45792, 45796, 45105, and 45417. In other embodiments, the lenses utilized in the system 100 are custom lenses with properties selected specifically for the various applications of the lenses within the system 100.

Further, in some embodiments the system includes an additional fiber optic cable (not shown) for addressing a lag time of a photodynamic therapy vector. This additional fiber optic cable is considered a lagging detector in some instances. In one particular embodiment, the lagging detector is utilized for detecting phosphorescence signals. In many instances, the confocal/adaptive optics system returns the majority of light back to the same point source of light (in system 100 that is the optical fiber 110). However, for certain modalities, such as phosphorescence, the light return is delayed in time. Accordingly, if focal point of the system is swept across the region of interest at a constant speed through the tissue, the time delay in the phosphorescence results in a position offset when received back at the fiber. Accordingly, using the secondary optical fiber at an offset position the delayed phosphorescence signal is received and detected. In some embodiments, the position of the secondary fiber can be moved dynamically relative to the primary fiber in order to compensate for the speed and direction of the focal point as it is swept across the region of interest.

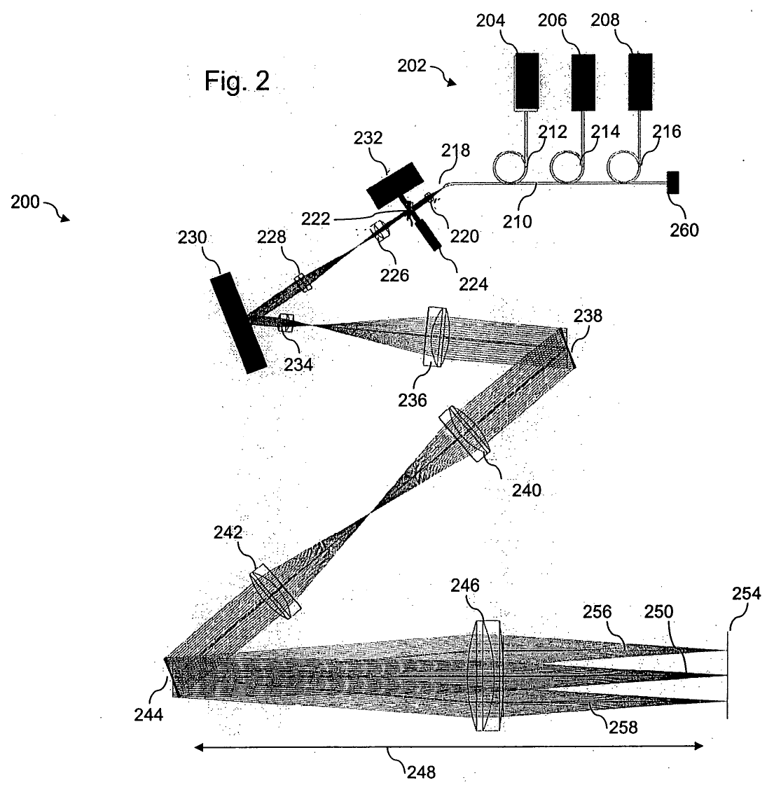

Referring now to FIG. 2, shown therein is a macroscope system 200 according to another aspect of the present disclosure. The system 200 includes an energy source 202. The energy source 202 may be substantially similar to that described above with respect to system 100. In that regard, the energy source 202 is utilized by the system 200 for both imaging purposes as well as treatment purposes. In the current embodiment, the energy source 202 includes a plurality of lasers 204, 206, and 208. Each of the lasers 204, 206, 208 comprises a different wavelength of light. In other embodiments, the energy source 202 includes more or less lasers. In some embodiments, the energy source 202 includes a single laser. In lieu of or in addition to the lasers 204, 206, and 208 the energy source 202 comprises one or more LEDs in other embodiments. In some embodiments, lasers with a wavelength between 550 nm and 1550 nm are used. In some embodiments, at least one of the lasers includes a wavelength of approximately 850 nm. In some embodiments, at least one of the lasers includes a wavelength of approximately 632 nm. In one particular embodiment, at least one of the lasers comprises a HeNe laser having a wavelength of approximately 632 nm. The specific wavelengths chosen and the number of different wavelengths chosen for the energy source 202 are selected based on the photodynamic therapy vectors in some instances. It is contemplated that any number and combination of energy sources may be utilized based on the photodynamic therapy vectors utilized.

Each of the lasers 204, 206, 208 are optically connected to a fiber optic cable 210 via connectors 212, 214, 216, respectively. In the current embodiment, the fiber optic cable 210 is a single-mode fiber. In some embodiments, the single-mode fiber has a core diameter between 1 μm and 10 μm. In other embodiments, a multi-mode fiber is used. Connectors 212, 214, 216 are optical connectors utilized to introduce the laser light emitted from the lasers 204, 206, 208 into the fiber optic cable 210. In the present embodiment, the connectors 212, 214, 216 are optical circulators. In other embodiments, one or more of the connectors 212, 214, 216 include other types of optical connectors or switches. Generally, any connector cable of introducing the energy from the energy source 202 into the cable 210 may be used in some embodiments. The lasers 204, 206, 208 are connected the fiber optic cable 210 such that one or more of the lasers may introduce light in to the cable. That is, a single laser may be activated or multiple lasers may be activated simultaneously. The determination of which lasers are to be activated and when is driven by such factors as the photodynamic therapy vectors being utilized, whether imaging or treatment is being performed, physician preference, and/or other factors.

After the light has been introduced into the cable 210 from the energy source 202, the light is emitted from the end 218 of the fiber optic cable. In some embodiments, the light is emitted from the fiber optic cable 210 with a half angle between approximately 20-30 degrees. The light emitted from the fiber optic cable 210 then passes through a lens 220 before reaching a splitter 222. In some embodiments, the splitter 222 comprises a beam splitter. Further, in some instances the beam splitter comprises a polarizing beam splitter. In other embodiments, the splitter 222 comprises a pickoff mirror. Further, in some instances the pickoff mirror picks off between 1-10% of the light passing therethrough. As the light passes through the splitter 222, some of the light is directed to a beam dump 224. The beam dump 224 absorbs the light that is directed its way. In that regard, the beam dump 224 may be utilized to reduce or eliminate any unwanted reflection from the energy source 202.

The remainder of the light passing through the splitter 222 continues on through a lens 226 and a lens 228 before reaching an adaptive optic device 230. In some embodiments, the adaptive optic device 230 comprises a deformable mirror. In other embodiments, the adaptive optic device 230 comprises a MEMS device. Generally, the adaptive optic device 230 is utilized to reduce the effects of optical distortion caused by human tissue, defects in the lenses/mirrors of the system 200, and/or other factors by measuring or monitoring the distortion caused by the tissue and rapidly compensating for it. In this manner, the adaptive optic device 230 along with the other components of the system 200 are utilized to minimize the scattering of light within human tissue, increase the penetration depth of the light, and increase drug efficacy while simultaneously reducing the required illumination intensity to activate the drugs. The adaptive optic device 230 is controlled at least in part by the wavefront detector 232. The wavefront detector 232 detects the distortion by monitoring the light reflected back through the system. In some embodiments, the wavefront detector 232 is a Shack-Hartmann sensor. Based on the detected distortion of the wavefront detector 252, an appropriate correction to the adaptive optic device 230 (e.g., adjustment of the deformable mirror) can be determined. In some embodiments, a computer (not shown) connected to the waverfront detector 232 is utilized to calculate an optimum mirror shape for the adaptive optic device 230 based on the detected distortion. The computer then directs the deformable mirror to reshape itself accordingly. In this manner the adaptive optic device 130 may be continuously updated to accommodate for the detected distortion. In some embodiments, the adaptive optic device 230 is updated in an interval between 1-10 milliseconds. In some embodiments, the adaptive optic device 130 and the system 100 in general are configured to operate utilizing a pulse-echo mode. In other embodiments, the system is configured to operate in a continuous wave mode. Accordingly, the energy source 102 and the lasers 104, 106, and 108 are configured for emitting light in a pulse mode and/or a continuous wave mode in some embodiments.

The light is reflected off of the adaptive optic device 230 through a lens 234 and a lens 236 before reaching a tip mirror 238. The tip mirror 238 is utilized to at least partially control the position of the light beam emitted from the system 200 in an x-y plane. In some instances, the tip mirror 238 is controlled by a computer (not shown) in communication with the adaptive optic device 230 and the wavefront detector 232. After being reflected off of the tip mirror 238, the light continues through lenses 240 and 242 to a tilt mirror 244. The tilt mirror 244 is utilized to at least partially control the position of the light beam emitted from the system 200 in the x-y plane. In some instances, the tilt mirror 238 is controlled by a computer (not shown) in communication with the adaptive optic device 230, the wavefront detector 232, and/or the tip mirror 238.

After being reflected off of the tilt mirror 244, the light continues through a lens 246. The lens 246 controls the depth of a focal point 254 of the beam 250 as it exits the system 200. In that regard, the tip and tilt mirrors 238, 244 may be understood to control the position of the focal point 254 of the beam 250 in an x-y plane (as indicated by alternate beams 256 and 258), whereas the lens 246 is utilized to control the depth of the focal point 254 along a z-axis perpendicular to the x-y plane. In that regard, the lens 246 may be moved or translated along an axis 248 parallel to the z-axis to control the depth of the focal point 254. In some embodiments, the lens 246 is translated by an actuator (not shown) that is controlled by the computer, which is also in communication with the adaptive optic device 230, the wavefront detector 232, the tip mirror 238, and/or the tilt mirror 244.

The light reflects back from the focal point 254 through the system 200. In particular, the reflected light passes through the lens 246, reflects off of the tilt mirror 244, passes through lenses 242 and 240, reflects off of the tip mirror 238, passes through lenses 236 and 234, reflects off of the adaptive optic device 230 and through lenses 228 and 226 before reaching the splitter 222. At the splitter 222, a portion of the reflected light is directed to the wavefront detector 232. In some embodiments, a small portion of the reflected light is also directed to the beam dump 224. In other embodiments, none of the reflected light is directed to the beam dump 224. The remainder of the reflected light continues through lens 220 and back into the optical fiber 210 where it continues to an optical detector 260. The reflected light sensed by the optical detector is utilized to form the images of the patient's tissue for diagnosis and/or treatment purposes. In some embodiments, the optical detector 260 is in communication with a computer for producing 2-D and 3-D renderings of the detected images. In some embodiments, the optical detector 260 is a high speed optical detector.

In some embodiments the reflected light detected by the optical detector 260 is screened to better clarify the resulting images. For example, in some embodiments the optical detector 260 is configured such that it accepts a majority of the ballistic reflected light and rejects a majority of the multiply-scattered reflected light. In that regard, the timing of the optical detector 260 is coordinated with the emission of light from the energy source 202 such that the optical detector accepts reflected light only within a predetermined time period after the emission of the light. The predetermined time period is chosen such that it coincides with the timing of the ballistic reflected light reaching the optical detector 260. In some embodiments, the predetermined time period is determined by a computer in communication with the optical detector 260, adaptive optic device 230, wavefront detector 232, tip mirror 238, tilt mirror 244, and/or lens 246. Reflected light received outside of the predetermined time period, which may consist of a substantial portion of the multiply-scattered reflected light, is rejected or ignored by the optical detector 260 and/or the computer utilized for producing the images based on the reflected light received. In one particular embodiment, the emission of light from the energy source 202 is between approximately 0.5-5.0 picoseconds and the associated predetermined time period is between approximately 1-10 nanoseconds. While the term predetermined has been used to describe the time period it is understood that this is merely exemplary and should not be considered limiting. For example, in some embodiments an appropriate time period is determined in near real time. In other embodiments, the time period may be determined after reception of the reflected light and taken into account when producing the image.

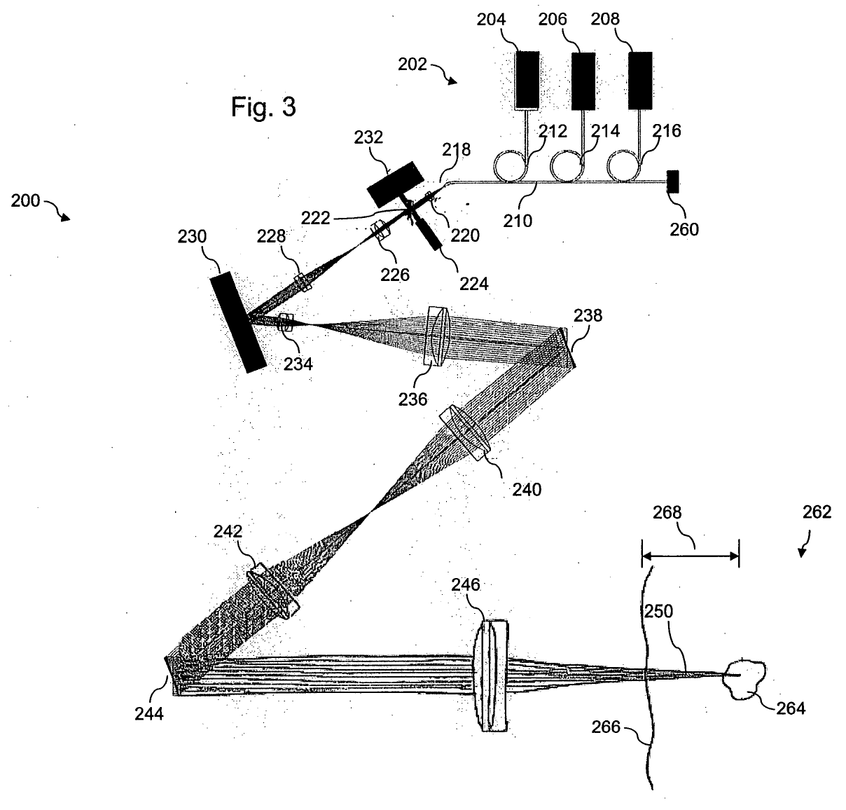

Referring now to FIG. 3, shown therein is the macroscope system 200 in use with a patient 262 having a cancerous region or tumor 264, as shown. The tumor 264 is positioned below a surface 266 of the patient 262 by a depth 268. In some instances, the tumor 264 is below the surface 266 at a depth between 50 microns and 20 mm. In other instances, the tumor 264 is below the surface 266 at a depth greater than 20 mm. In yet other instances, the tumor 264 begins substantially adjacent the surface 266 and extends below the surface. Accordingly, the macroscope system 200 is utilized to treat both surface and subsurface regions.

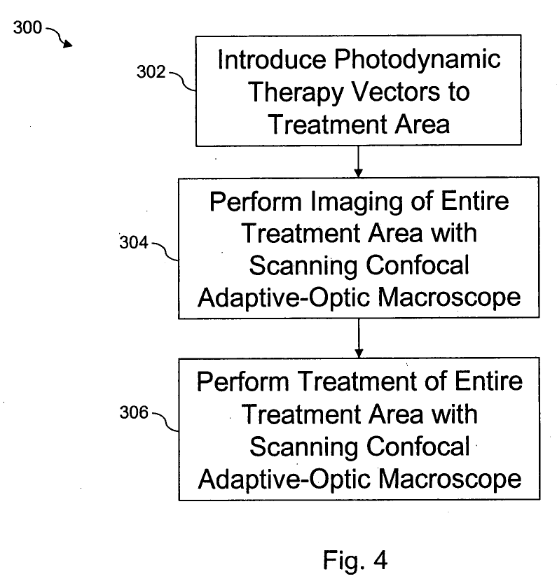

Referring now to FIG. 4, shown therein is a flowchart illustrating a method 300 of performing a photodynamic therapy according to one aspect of the present disclosure. The method 300 begins at step 302 with the introduction of a photodynamic therapy vector to the treatment area. It should be noted that while the term treatment area is used, this is not to be considered limiting. Rather, it is understood that the treatment area includes a volume of space in some instances.

Embodiments of the present disclosure are capable of imaging and activating all known photodynamic therapy vector and marking materials such as the photodynamic therapy drug Photofrin, the nanoparticle gold colloids, and/or the nanophosphors made by Authentix, Inc. Unlike the other materials, the nanophosphors' medical efficacy has a non-linear function relative to the photon density of illumination. In particular, if the intensity at the focal point is increased by a factor of 10, the treatment efficacy is subsequently increased by more than a factor of 10. Hence, with the more effective use of the excitation light by the systems of the present disclosure, the overall laser power of the treatment phase may be significantly reduced while still achieving the same biological effect. In addition to limiting the number of healthy cells killed, reducing the overall laser power during the treatment phase also reduces the health risks associated with unnecessarily overexposing patients to lasers.

Nanophosphors can also be illuminated with wavelengths of light that are very penetrative of human tissue, e.g. light with a wavelength of approximately 850 nm. While 850 nm wavelength light typically does not activate photosensitizer drugs, the nanophosphors can be activated by such light. The nanophosphors in turn can be utilized to downshift the wavelength of the light to an emission wavelength that secondarily activates photosensitizer drugs. Accordingly, in some embodiments a penetrative laser light is introduced to activate a first phototherapy vector (such as a nanophosphor), which in turn activates a second phototherapy vector (such as Photofrin).

Gold nanoparticles include unique properties such as Surface-enhanced Raman Scattering (SERS) and Surface Plasmon Resonance (SPR) that may be utilized in the imaging and treatment processes. Use of optical detectors in conjunction with SERS an SPR modes require measurement of minute wavelength shifts (both Stoke and anti-Stokes shifts) that allow for strong detection of these particles. To detect these small shifts, the system may include fiber optic telecom technologies such as fiberized wavelength sources, detectors, filters, switches, or otherwise.

At step 302, a medical personnel administers the photodynamic therapy vector to the patient in the manner specified by the particular vector. In a majority of embodiments, the photodynamic therapy vector introduced will preferentially cling (to some degree) to the cancerous lesions, cells, or tumors. Depending upon the photodynamic therapy vector there may be a delay in time required to allow the convergence of the vector to the cancerous region.

After the photodynamic therapy vector has been introduced at step 302, the method 300 continues with step 304 in which the treatment area is imaged with a scanning, confocal, adaptive-optic macroscope. In some embodiments, step 304 includes the medical personnel opening a software suite for controlling the macroscope system and positioning the optical head of the system relative to the treatment region of the patient. In some embodiments, at least a portion of the macroscope system is positioned within an articulated arm system to allow easy positioning of the optical head or output of the system over the desired region of the body as needed. Then, the macroscope system is energized and a two part operation of the system is started. The first part is the imaging step 304; the second part is a therapy or treatment step 306.

In some embodiments, the imaging step 304 comprises creating a 3-D image of the treatment region. In some instances the 3-D image is at least partially based on detecting the photodynamic therapy vectors within the treatment area. In that regard, the attraction or bonding of the photodynamic therapy vectors to the cancer cells may be detected using the confocal macroscope system. In that regard, such factors as the luminescence, absorption, scattering, and/or other detectable factors of the reflected energy may be utilized in forming the 3-D image. In some embodiments, the imaging step 304 also includes identification of cells or regions that are to be treated and cells or regions that are not to be treated. In that regard, this determination may be made by medical personnel or a computer based on a set of associated rules.

After the imaging step 304 is completed, the method continues at step 306 with treatment of the treatment area. In that regard, those regions identified in step 304 as needing treatment are subjected to treatment, whereas the other regions are not treated. In this manner, the number of healthy cells killed by the process is limited. Treatment of the various regions includes moving the focal point of the macroscope to each of the associated treatment regions. Such movement may be accomplished by modifying the tip and tilt mirrors and/or the focus mirror of the macroscope system. In other instances, the movement is accomplished by precisely moving an articulated arm system associated with the macroscope. In some instances, the movement of the focal point is computer controlled. Unlike the imaging step 304, the treatment step 306 has an incident light of much higher power. This level of laser power kills the cells at the high intensity focal point within the tissue. However, due to the highly focused nature of the light, areas nearby receive a much more diffuse light that does not cause mortality. In some embodiments, the treatment step also includes a feedback system. In that regard, the feedback system constantly monitors for the photodynamic markers. If no markers are found (as in the case of a patient moving or incorrect placement of the optical head), the laser does not fire. In some embodiments, the treatment step 306 of the method 300 may be performed without performing the imaging step 304. In that regard, the treatment step may be performed by relying on feedback from the in-vivo markers. However, there is a great deal of contextual information, such as density of particles or distribution of particles, in the 3D mapping that is desirable in some instances.

In most embodiments, the treatment step 306 relies on the 3D map or image of the treatment area and the cancerous regions marked by step 304. In particular, the power of the laser is selectively increased and, with the help of the 3D map, applied only to those cells marked with the photodynamic therapy drugs or markers. This allows healthy regions to be avoided. It should be noted that each of the imaging step 304 and the treatment step 306 may be repeated as necessary for completing treatment of the cancerous region. This permits the physician to switch between the imaging and treatment modes of the macroscope as desired during the procedure. This enable updates to the map and complete coverage to the marked regions, including during the treatment phase. This detection and feedback loop allows for exclusive excitation (and mortality) of cancerous cells with high intensity illumination. Further still, an accurate 3D map of the marked cancerous area enables the physician to return to any region at any point in time without doing damage to the surrounding tissue.

The particular wavelengths, intensities, and other parameters of the imaging and treatment steps are dependent upon such factors as the photodynamic therapy vector used, location of the cancerous region, and/or other factors. Specific examples of employing various photodynamic therapy vectors will now be provided, but are in no means limiting to either the system or the method of using the system with each of the photodynamic therapy vectors. Further, the systems of the present disclosure are configured for use with other photodynamic therapy vectors not discussed herein in other embodiments.

Using FDA approved Photofrin, a three dimensional fluorescence image of the tumor area is obtained using the macroscope system. In some instances, this imaging requires a very low power excitation laser having a first wavelength and a detector for detecting a different wavelength. Once the 3D image of the tumor has been imaged, the medical personnel may either automatically allow the device to destroy the marked tissues identified as being cancerous and/or use the 3D “map” of the tumor, and mark out regions for destruction. The device then increases the laser power to a “moderate” level and point-by-point excites the Photofrin cells. The precision of the confocal macroscope reduces the number of healthy cells killed as compared to broader illumination of typical photodynamic therapy treatments that kill both healthy and cancerous cells. Further, because there is no need for an extended time delay to allow for dosage leakage, much less of the phototoxic drug is introduced into the patient initially as compared to typical photodynamic therapy treatments. Further, in some embodiments the markers are utilized to mark healthy cells, but not the cancerous cells. In such an embodiment, the macroscope system is utilized to kill the unmarked cancerous cells.

Using gold nanoparticles as the photodynamic therapy vector instead of Photofrin, a similar method is undertaken. However, for the absorbing gold nanoparticles, in some embodiments two different wavelengths of laser are used simultaneously during the three dimensional imaging. In such an embodiment, the ratio of back scattered intensity is utilized to map out the gold nanoparticles—one wavelength is absorbed by the gold nanoparticles while the other is scattered. The scattering and absorption information is utilized to build up the 3D image point-by-point. Then, just as with Photofrin, the laser intensity is raised to a moderate level and scanned over the treatment area point-by-point exciting the gold nanoparticles of the cancerous region.

Using nanophosphors instead of the Photofrin or gold nanoparticles, imaging may be performed in several ways, including up or down wavelength shifting (fluorescence or up-conversion), time delay, pulse response, and/or other imaging techniques. Again, a 3D image of the tumor is created point-by-point using one of the selected imaging methods. Then the medical personnel decide what areas to treat with the higher intensity laser and what areas to leave alone. In some embodiments, the medical personnel marks the portions of the 3D image to be treated. Then, based on the marked-up 3D map of the tumor, the macroscope system fires the correct laser to either heat the nanoparticles or turn on the nearby PhotoFrin point-by-point for each marked point.

In some instances there is an uneven, undesirable, or inaccurate distribution of the photodynamic therapy vector within the tissue of the treatment area. For example, in some instances Photofrin or gold nanoparticles are found in healthy tissue. These potential problems are overcome by embodiments of the present disclosure with the precise treatment and image guided approach to therapy. By treating only those areas of tissue with at least a certain threshold level of Photofrin, or only those nanoparticle-marked areas that form a surface (as opposed to loose, individual gold nanoparticles) these problems are avoided. Further, as discussed above the treating physician or medical personnel defines the treatment regions in the 3-D image in some instances. Accordingly, the treating physician or medical personnel can select only the desired treatment regions and ignore healthy areas that happen to include some of the photodynamic therapy vector. Further, in some embodiments the imaging detectors remain operational during the treatment phase. This allows for near real-time checking that the areas that are being treated either have the desired marker present and/or was selected by the medical personnel as an area to be treated. Accordingly, if the patient shifts slightly so that the image map and the treatment region no longer corresponds to one another, the medical personnel can be quickly and automatically notified, ensuring safe operation of the system and preventing unwanted treatment to non-cancerous areas. Further, this option may be utilized for an automatic treatment where the device scans the treatment area and according to preset thresholds or rules, the laser power is automatically increased to destroy the cancer tissue based on the rules.

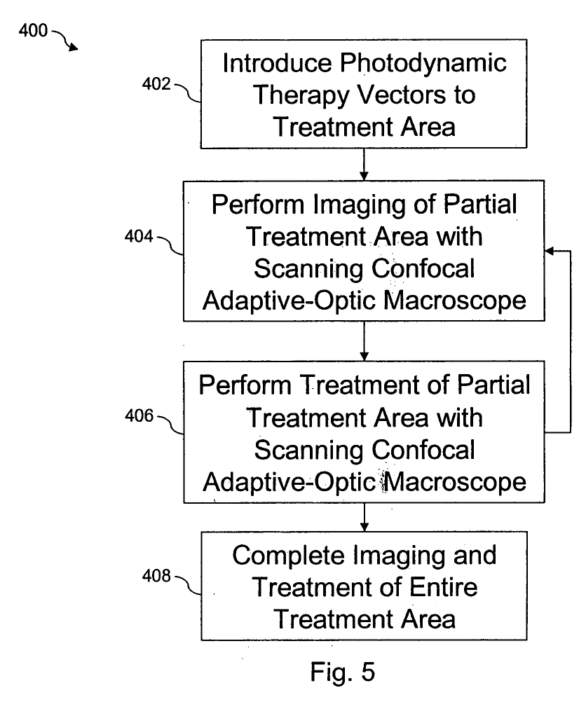

Referring now to FIG. 5, shown therein is a flowchart illustrating a method 400 of performing a photodynamic therapy according to another aspect of the present disclosure. The method 400 is substantially similar to the method 300 described above with respect to FIG. 4 in many respects. For example, the method 400 begins with step 402 in which photodynamic therapy vectors are introduced to the treatment area. Again, a medical personnel administers the photodynamic therapy vector to the patient in the manner specified by the particular vector. In a majority of embodiments, the photodynamic therapy vector introduced will preferentially cling (to some degree) to the cancerous lesions, cells, or tumors. Depending upon the photodynamic therapy vector there may be a delay in time required to allow the convergence of the vector to the cancerous region.

The method 400 continues with step 404 in which a portion of the treatment area is imaged using a scanning, confocal, adaptive-optic macroscope. After the imaging step 404, the method 400 continues with step 406 in which the portion of the treatment area imaged in the previous step is either treated or not treated depending on a determination of whether the portion of the treatment area comprises cancerous cells or healthy cells. In some embodiments, the determination of whether to treat the portion of the treatment area (i.e., subject to increased laser intensity to activate the photodynamic therapy vectors) is performed by medical personnel. In other embodiments, the determination is made a computer system according to a set of rules for identifying cancerous cells. In some instances the rules are at least partially based on the concentration (or lack thereof) of photodynamic therapy vectors in the cells. If the determination is made to treat the portion of the treatment area, then the laser intensity, wavelength, or other energy source factor is modified according to the photodynamic therapy vectors in use to treat that portion of the treatment area.

After treatment, the method 400 continues back a step 404 where another portion of the treatment area is imaged, after which that portion is either treated or not treated at step 406. This iteration continues until the entire treatment area has been imaged and treated as needed at step 408 of the method 400. Using this approach in a real time manner, every portion of the treatment area is scanned at low intensity, determined if there are any photodynamic therapy vectors present and, if so, the power of the laser is increased to a treatment level for that portion exclusively before moving to the next portion. In some embodiments, the portions of the treatment area considered during step 404 overlap such that a majority of the points within the treatment area are considered for treatment more than once. In some embodiments, the scanning, confocal, adaptive optic macroscope systems of the present disclosure are utilized in performing the method 400. In particular, the ability of the macroscope systems of the present disclosure to perform the two functions—imaging and in-situ treatment of marked cancerous region—makes them particularly well suited for these methods. During such a medical procedure, the macroscope systems of the present disclosure have an ability to rapidly switch between these two modes: viewing the cancerous cells before actually targeting and treating them and vice versa.

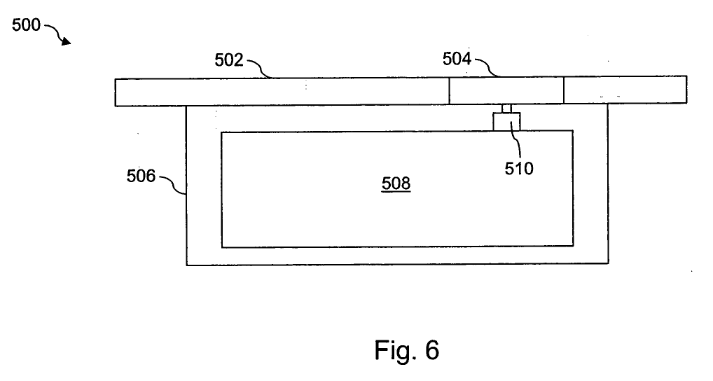

Referring now to FIG. 6, shown therein is a patient treatment table 500 according to one aspect of the present disclosure. The patient treatment table 500 includes an upper table portion 502 for supporting a patient. In particular, the table 500 is sized to allow a patient to sit or lay down on the table. In that regard, the table 500 includes a window 504 within the upper portion 502. In use, the treatment area or area of interest on the patient is positioned adjacent to the window 504. In some embodiments the window 504 comprises a glass panel providing visual access to a lower portion 506 of the table 500. The lower portion 506 of the table 500 houses an imaging and treatment device 508. In some embodiments, the imaging and treatment device 508 is a scanning, confocal, adaptive-optic macroscope system according to aspects of the present disclosure. In that regard, the output or optical head of the device 508 is positioned adjacent to the window 504. In some embodiments, the window 504 is part of the imaging and treatment device 508. In other embodiments, the window 504 is separate from the imaging and treatment device 508, but the imaging and treatment device takes into account the optical effects of the window.

While the patient treatment table 500 may be used for treating many types of medical conditions, including numerous oncology applications, it has particular application to the treatment of breast cancer in some instances. For example, in some embodiments the treatment table 500 is particularly adapted for treating ductal carcinoma in situ. In that regard, the table 500 and, in particular, the upper portion 502 is configured for a patient to lay face-down such that the patient's breasts are positioned adjacent to the window 504. In some instances the table further includes a device to limit movement of the patient's breasts relative to the table during imaging and treatment. In one embodiment, the table includes a suction device or vacuum opening adjacent to the window 504. The suction or vacuum device is utilized to hold the skin adjacent to the patient's breasts substantially fixed relative to the table to thereby limit the movement of the patient relative to the window 504. Further, by having the patient lay face-down on the table movement of the patient's breasts caused by breathing is substantially limited.

With the patient substantially stationary relative to the window 504, the imaging and treatment device 508 is utilized. In some embodiments, the imaging and treatment device 508 is used substantially in the manner described above with respect to other embodiments of the present disclosure. For example, the imaging and treatment device 508 is utilized in conjunction with photodynamic therapy vectors in some instances. The imaging and treatment device 508 is utilized to created a 3-D image or map of the region of interest and then precisely kill the identified cancerous cells. In some instances the table 500 is used solely for imaging purposes. In other instances the table 500 is used solely for treatment purposes. In some embodiments, the table 500 is utilized in conjunction with other imaging device to identify the location of the cancerous cells.

Embodiments of the present disclosure substantially reduce the risk of complications associated with other oncology treatment options, such as surgery (recovery, infection, anesthesia, etc.), chemotherapy, and radiation. The present disclosure allows for oncology treatments to be conducted in a physician's office, eliminating the need for an expensive operating space and staff. This is not only cost-efficient, but also time efficient. Further, the level of laser light intensity is dramatically less than the amount used in conventional photodynamic therapy in some instances. Because of the scanning confocal adaptive-optic macrocsope systems of the present disclosure, there is significantly less internal scattering and the incident light is focused more precisely only on those marked areas. Accordingly, much less light is needed overall to activate the photodynamic therapy vectors (reducing adverse patient reactions), yet the targeted areas have a higher concentration of light increasing the efficacy of the photodynamic therapy vectors in the marked areas. With the reduced levels of laser intensity and with its noninvasive nature, embodiments of the present disclosure require a local anesthetic in some photodynamic therapy treatments.

Further, the present disclosure allows the photosensitizing process to use of a significantly lower quantity of drug (e.g. Photofrin) and by treating immediately rather than waiting the currently prescribed 24-72 hours. In some embodiments, the present disclosure allows surface treatments immediately following introduction of the photodynamic therapy vector. The reduction in the quantity of applied drugs significantly reduces the heightened light sensitivity and toxicity to the patient compared to existing methods. As the treatment occurs immediately following the application of the drug, the concentration of Photofrin in the cancerous regions is equal to what would have been under the higher concentrations after the delay. While both healthy cells and cancerous cells are approximately equally dosed, the equal dosage is of limited consequence to the healthy cells because only those cells identified as cancerous by the macroscope system and/or the physician are destroyed. Accordingly, the overall effect of one aspect of the present disclosure is a significantly lower need for photodynamic therapy vectors, faster recovery time, and less swelling of healthy tissue.

Where other photodynamic therapy treatment techniques are limited in depth, embodiments of the present disclosure intentionally introduce optical aberrations to equal and cancel the optical aberrations of imaging within tissue that would otherwise scatter the incident light away from the target preventing accurate imaging and/or treatment. This laser wavefront control achieves the required depth for in-vivo imaging and treatment. Whereas other methods equally illuminate (and damage) both healthy and cancerous regions, embodiments of the present disclosure differentiates the healthy from the diseased using a lower power laser for imaging, and only destroys the cancerous regions using a higher power laser after specifically targeting those cells. In that regard, embodiments of the present disclosure concentrate the laser power onto the marked cancerous cells, while intentionally missing both the bound and unbound healthy cells resulting in a substantial reduction of healthy cell mortality.

The adaptive-optic confocal macroscope systems of the present disclosure are first used to scan the entire region of interest at a very low power level, building up a very detailed, point-by-point, 3D image of the region. In some embodiments, the low level of intensity of the laser during imaging does not affect the cancer cells, the markers, or the healthy cells. During this imaging procedure, the physician or other medical personnel is able to locate and identify the cancer cells in real time, without the need for X-rays, MRIs, or other typical imaging procedures and without the need for invasive components or procedures. The present disclosure also provides for the non-invasive treatment of cancerous cells or tumors within the patient after the imaging process. The non-invasive treatment substantially eliminates the need for patient incision and/or general anesthesia in most instances. The combination of non-invasive imaging and treatment allows for oncology treatments utilizing embodiments of the present disclosure to be carried out in a doctor's office, rather than an operating room.

In some embodiments, macroscope systems of the present disclosure are configured to excite two photon processes within deep tissue. Like upconversion, two photon processes accept two low power (but penetrative) photons and generate one high power photon, that activates a photodynamic therapy process. While all organic materials exhibit some level of two-photon effect, most organic processes are much less efficient than nanophosphors. The reason for this is the low effective “absorption cross section” of these organic two-photon effects. To get a noticeable effect, a high degree of photon density must occur. Whereas typical activating lasers have been limited to surface treatments for this reason, embodiments of the present disclosure are able to provide sufficient photon density into deeper tissues.

While Photofrin is a well known photodynamic therapy vector, it is usually not utilized in a detection (presence/absence) mode. Rather, it is applied, a time delay occurs, and then the general area to be treated is illuminated. As previously noted, because of some residual PhotoFrin in healthy cells these are also damaged. Embodiments of the present disclosure, however, have the capacity to directly image the Photofrin within the patient's tissue. Using only this information, the present invention may then excite the Photofrin only in areas of high concentration (cancerous sites) while ignoring low concentration areas (healthy cells). This specific excitation allows for more accurate laser usage leading to lower mortality of healthy cells.

In some embodiments, the present disclosure is used in conjunction with white light oncology treatments. White light oncology allows for physician-based decision making and treatment without any markers. Hemoglobin and other tissues have wavelength absorbances, and the use of multiple wavelengths allows for 3D image formation of any tumors or irregular cells. With the physician's guidance, embodiments of the present disclosure are utilized first to image the tumors with a first wavelength of light and then switch wavelengths to an absorbing color and blast those regions.

While embodiments of the present disclosure have been described primarily with respect to deep tissue or below the surface treatments, in some instances embodiments of the present disclosure are utilized for surface treatments (such as treatment of basal cell carcinoma). Further, in addition to the standard photodynamic therapy surface treatments, the macroscope systems of the present disclosure are also utilized to remove birthmarks, hemangiomas, or tattoos from the skin surface.

Further, in some embodiments the macroscope systems of the present disclosure are utilized for laser pulse induced sonication. Laser pulse induced sonication requires a short pulse from a high-power laser focused to a very tight spot within a cell system. In some instances the pulse is tuned so as to not do any long-lasting damage to the cell. Rather, the pulse of energy causes an otherwise non-porous cells to become temporarily porous, allowing treatment-related agents such as drug vectors, particles, etc. to enter the cells.

Further, in some embodiments the macroscope systems of the present disclosure are utilized for photocoagulation. Photocoagulation is the coagulation (clotting) of blood or tissue using light. In some embodiments, green laser light is selectively absorbed by hemoglobin, the pigment in red blood cells, in order to seal off bleeding blood vessels. Photocoagulation has diverse uses including as cancer-related treatment. For example, photocoagulation can destroy blood vessels that lead to rapidly growing tumors. It can also be used in the treatment of pathologies related to the visual system, such as targeting a detached retina, destroying abnormal retinal blood vessels, treating eye tumors, or otherwise.

Further, in some embodiments the macroscope systems of the present disclosure are utilized for photostimulation. Photostimulation is the use of light to artificially activate biological compounds, cells, or even whole organisms. By using only light, photostimulation can be used to non-invasively probe the causal relationship between different biological processes. Photostimulation may be useful as a form of light therapy. Photostimulation methods fall into two general categories: one set of methods uses light to uncage a compound that then becomes biochemically active, binding to a downstream effector. For example, releasing glutamate is useful for finding excitatory connections between neurons, since the neurotransmitter mimics the natural synaptic activity of neuronal interaction. A second major photostimulation method is the use of light to activate light-sensitive proteins such as rhodopsin, which can then excite the cell expressing opsin, which is a vital process of the visual system. It has been suggested that channelrhodopsin—a monolithic protein containing a light sensor and an ion channel—provides stimulation of appropriate speed and magnitude.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

What is claimed is:

1. A macroscope system for use in deep tissue photodynamic therapy, comprising:
an energy source configured for providing a point source of energy having a first characteristic for imaging the deep tissue and providing a point source of energy having a second characteristic for treating the deep tissue;
an adaptive optic device in optical communication with the point source of energy; and
an optical head portion in optical communication with the point source of energy, the optical head portion comprising a focal lens moveable along an optical axis for adjusting a depth of a focal point of the energy within the deep tissue;
wherein the adaptive optic device is configured to at least partially correct for aberrations caused by tissue between the focal lens and the deep tissue.

2. The system of claim 1, wherein the energy source comprises a light source and provides a point source of light.

3. The system of claim 2, wherein the point source of light is emitted from a first fiber optic cable.

4. The system of claim 3, wherein the energy source comprises a plurality of laser light sources, at least two of the plurality of laser light sources emitting different wavelengths of light.

5. The system of claim 4, wherein at least two of the plurality of laser light sources are operable simultaneously.

6. The system of claim 3, further comprising a lagging detector for detecting phosphorescence signals from the deep tissue, the lagging detector comprising a second fiber optic cable offset from the first fiber optic cable.

7. The system of claim 3, wherein at least two of the plurality of laser light sources are configured for use with different photodynamic therapy vectors.

8. The system of claim 2, further comprising an optical detector system for receiving light reflected from the focal point.

9. The system of claim 8, wherein the optical detector system is configured to receive light of different wavelengths and separate the light into a plurality of channels based on the different wavelengths.

10. The system of claim 2, wherein the depth of the focal point of the energy within the deep tissue is at least 500 microns below a tissue surface.

11. The system of claim 1, wherein the optical head portion comprises:
a static negative optical element;
a static positive optical element in optical communication with the static negative optical element;
an aperture in optical communication with the static positive optical element; and
a dynamic positive optical element in optical communication with the aperture, the focal lens being the dynamic positive optical element.

12. The system of claim 1, wherein the first characteristic is a first intensity and the second characteristic is a second intensity, the second intensity being greater than the first intensity.

13. The system of claim 12, wherein the second intensity is sufficient to activate a photodynamic therapy vector.

14. The system of claim 1, wherein the first characteristic is a first wavelength and the second characteristic is a second wavelength, the second wavelength being different than the first wavelength.

15. A method of performing deep tissue photodynamic therapy, the method comprising:
introducing a photodynamic therapy vector into a region of interest within a deep tissue of a patient;
imaging the region of interest with a scanning, confocal macroscope to facilitate identification of one or more cancerous portions within the region of interest; and
activating the photodynamic therapy vector within the one or more cancerous portions of the region of interest with the scanning, confocal macroscope.

16. The method of claim 15, wherein the imaging comprises forming a 3-D image of the region of interest and wherein the scanning, confocal macroscope utilizes adaptive optics to correct aberrations caused by the patient's tissue.

17. The method of claim 16, wherein the facilitating identification identifies the one or more cancerous portions and wherein the cancerous portions are marked on the 3-D image.

18. The method of claim 15, wherein the imaging comprises emitting a laser from the macroscope at a first level of intensity and the activating comprises emitting a laser from the macroscope at a second level of intensity, the second level of intensity being greater than the first level of intensity.

19. The method of claim 18, further comprising switching the macroscope from the imaging mode and the first level of intensity to the activating mode and the second level of intensity, wherein the switching is performed immediately upon identifying a portion within the region of interest.

20. A macroscope system for use in deep tissue photodynamic therapy, comprising:
a single-mode fiber optic cable for providing a point source of light;
a plurality of lasers optically connected to the fiber optic cable, each of the plurality of lasers configured for emitting a light of a different wavelength into the fiber optic cable;
an adaptive optic device in optical communication with the point source of light;
a tip mirror in optical communication with the adaptive optic device;
a tilt mirror in optical communication with the tip mirror;
an optical head portion in optical communication with the tilt mirror, the optical head portion for emitting a beam of light having a focal point within the deep tissue of a patient that is 500 microns to 10 mm below a surface of the patient, wherein the optical head portion comprises a focal lens moveable along an optical axis for adjusting the depth of the focal point of the energy within the deep tissue;
wherein the adaptive optic device is configured to at least partially correct for aberrations caused by patient tissue between focal lens and the deep tissue;
wherein the macroscope system is configured to image the deep tissue of the patient in a first mode and to excite a photodynamic therapy vector within the deep tissue of the patient in a second mode.
